(19) United States
(12) Patent Application Publication
(10) Pub. No.: US 2008/0107784 A1
(43) Pub. Date: May 8, 2008

(54) Process To Improve Activity Of Mannoprotein As Wine Stabiliser

(76) Inventors: Peter Philip Lankhorst, Rotterdam (NL); Ibrahim Ozkan, Rotterdam (NL)

Correspondence Address: NIXON & VANDERHYE, PC, 901 NORTH GLEBE ROAD, 11TH FLOOR, ARLINGTON, VA, 22203, US

(21) Appl. No.: 11/792,998

(22) PCT Filed: Dec. 20, 2005
(86) PCT No.: PCT/EP05/56964

§ 371 Date: Jun. 14, 2007

(30) Foreign Application Priority Data
Dec. 23, 2004 (EP) 04106947.7

(51) Int. Cl. C12H 1/14; C12P 21/00

(52) U.S. Cl. 426/271; 426/592; 426/656

(57) ABSTRACT

The present invention describes a process to improve the activity as wine stabiliser of a mannoprotein, comprising treating a mannoprotein with a basic solution at a pH of at least 9. The mannoprotein obtainable by this process is more effective in stabilising wine against tartrate precipitation or protein haze formation when compared with the mannoprotein prior to the treatment with basic solution.

### FIELD OF THE INVENTION

The present invention relates to a process to improve the activity of a mannoprotein as wine stabiliser, to a mannoprotein with improved activity obtainable by this process and to the use of said mannoprotein as wine stabiliser.

### BACKGROUND OF THE INVENTION

The presence of tartaric salts, potassium hydrogen tartrate (KHT), calcium tartrate (CaT) and the development of protein haze are major causes of instability of wines.

Tartaric acid is the main organic acid produced by the grape berry during its development. It is solubilised in the form of potassium and calcium salts into grape musts during the processing of berries. During the fermentation, the solubility of salts of tartaric acid decreases with the increase of ethanol concentration (due to the fermentation of sugars).

In young wines, potassium hydrogen tartrate (KHT) is always present in supersaturating concentrations and crystallises spontaneously. After bottling of wines, the KHT-instability may become a commercial problem due to the unpredictable character of the crystallisation. Besides, consumers often perceive the presence of crystals in the bottle as a sign of inferior quality of the wine. Physical treatments can be used prior to bottling of the wine to prevent crystallisation of tartrate salts. These treatments consist in promoting the crystallisation by cooling the wine to −4° C. or in elimination of the potassium and tartaric ions by electrodialysis or by the use of ion-exchange resins. However, these time- and energy-consuming processes are supposed to alter the colloidal equilibrium of wines.

The alternative to physical treatments of wines is to use additives, which prevent the nucleation and/or the growth of KHT crystals.

Carboxymethyl cellulose and meta-tartaric acid belong to the group of additives which inhibit the growth of KHT crystals. Unfortunately, carboxymethyl cellulose has not been accepted by the wine community due to its presumed negative organoleptic effect on treated wines. Meta-tartaric acid, on the other hand, is unstable at the pH of wine and at the temperature at which wine is stored. Over time, the meta-tartaric acid will hydrolyse and its protective effect will disappear. Therefore, its use is restricted to low quality wines for quick consumption. Another drawback is that ideally an additive should be a natural component of wine. This is definitely not the case with meta-tartaric acid or carboxymethyl cellulose.

Another cause of wine instability is the aggregation of unstable wine proteins, which gives rise to protein haze formation and which contributes to reduce the perceived wine quality. Currently, protein haze formation is removed from wine using bentonite. However this treatment has a negative effect on the organoleptic characteristics of wine. Furthermore this treatment requires additional work for the winemaker and leads to loss of wine, which remains absorbed by the bentonite.

Natural additives, which are active against protein haze formation and on both nucleation and growth rate of KHT crystals, are preferred to chemical additives.

An example of natural additive is mannoprotein. Mannoprotein is, together with glucan, the main component of cell walls in yeasts (Lipke P. N. et al, J. Bacteriol. (1998) 180(15): 3735-3740). Mannoprotein is mainly obtained from yeast cells by two types of methods: physical methods and enzymatic methods. The most common physical method to obtain mannoprotein is that described in Peat S. et al, J. Chem. Soc. London (1961) 28-35, wherein yeast is autoclaved at 138° C. for 2 hrs in citrate buffer and, after removal of the cell walls, the product is precipitated by addition of ethanol, further purified by dialysis and isolated by lyophilisation. Enzymatic methods to obtain mannoprotein from yeast are described in WO 96/13571 and in WO 97/49794, and essentially comprise treatment of isolated yeast cell walls with a β-glucanase preparation and isolation of the product via ultrafiltration.

Mannoprotein produced by the known methods, e.g. obtained by autoclaving, has the disadvantage that its effectiveness in stabilising wine, in particular in preventing nucleation and/or growth of KHT-crystals or in preventing protein haze formation is not always satisfactory.

There is therefore a need for a process to improve the activity of a mannoprotein as wine stabiliser. There is also a need for a mannoprotein with improved activity as wine stabiliser.

### DETAILED DESCRIPTION OF THE INVENTION

The present invention provides, in a first aspect, a process to improve the activity of a mannoprotein as wine stabiliser, comprising treating a mannoprotein with a basic solution at a pH of at least 9.

In the context of the present invention “the activity of a mannoprotein as wine stabiliser” refers to the ability of a mannoprotein, when added in sufficient amount to wine or to grape must to be used in the production of wine, to produce a stabilising effect in the treated wine when compared with the wine prior to the addition of mannoprotein (hereafter indicated as untreated wine). The stabilising effect can comprise preventing and/or retarding the crystallisation of salts of tartaric acid in the treated wine or preventing and/or reducing the formation of protein haze in the treated wine (when compared with the untreated wine). The stabilising effect due to the ability of a mannoprotein to prevent and/or retard the crystallisation of salts of tartaric acid can be determined at −4° C. and at a chosen concentration of mannoprotein in wine, by measuring the time (T_{crys}) necessary to visually observe the appearance of crystals of KHT in wine and comparing it with the T_{crys} measured under the same conditions in the untreated wine. The ability of a mannoprotein to prevent and/or reduce the formation of protein haze can be measured at a chosen concentration of mannoprotein in wine, by heating said wine at 80° C. for 6 hours, cooling at 4° C. and determining the turbidity of the wine by nephelometry (at 860 nm) and comparing it with the turbidity measured under the same conditions in the untreated wine.

To “improve” the activity of a mannoprotein as wine stabiliser means that when the mannoprotein obtainable by the process of the invention is added in a sufficient amount to produce a stabilizing effect, i.e. is added in a sufficient amount to increase the T_{crys} of the treated wine or to decrease the turbidity of treated wine when compared with the untreated wine, said stabilizing effect is higher (i.e. the T_{crys} of the treated wine is longer or the turbidity of the treated wine is lower) than the stabilizing effect achieved with the mannoprotein prior to the basic treatment used in the same amount.

In the context of the present invention “mannoprotein” defines a product which is derived from yeast and which can be identified, by standard analytical methods (such as amino acid analysis, carbohydrate analysis etc.), as a combination of a protein moiety and of a carbohydrate moiety comprising polymers of mainly mannose. The carbohydrate moiety and the protein moiety are not necessarily covalently bound to each other.

The mannoprotein used (as a starting material) in the process of the invention can be derived from yeast according to any method suitable thereto. Examples of these methods are among others described in Peat et al, WO 96/13571 and WO 97/49794.

The mannoprotein used in the process of the invention can also be obtainable by a process comprising: a) subjecting a suspension of yeast cells to enzymatic hydrolysis whereby said yeast cells are degraded and mannoprotein and other yeast components are solubilised and released from the degraded cells; b) recovering the solubilised mannoprotein.

In step a) a suspension of yeast cells is subjected to enzymatic hydrolysis whereby said yeast cells are degraded and mannoprotein and other yeast components are solubilised and released from the degraded yeast cells.

Any type of yeast can be used in the process of the invention. In particular, yeast strains belonging to the genera Saccharomyces, Kluyveromyces or Candida may be suitably used. Yeast strains belonging to the genus Saccharomyces, for example the strain Saccharomyces cerevisiae, are preferred.

The process to produce the mannoprotein to be used in the process of the present invention may start with a suspension of yeast cells in an aqueous liquid, e.g. a fermentation broth of the yeast cells in question. Suitable fermentation processes leading to suspensions of yeast cells are known in the art. In some cases the fermentation broth can be concentrated before use in the present process, for example by centrifugation or filtration. For example, cream yeast (Baker's yeast which has been concentrated to 15-27% w/w of dry matter content) may be used.

In step a) the enzymatic hydrolysis of the suspension of yeast cells may be performed by subjecting said suspension to the action of native yeast enzymes and/or added exogenous enzymes.

The conditions used to perform the enzymatic hydrolysis are dependent on the type of enzyme used and can be easily determined by those skilled in the art. Generally, enzymatic hydrolysis will be performed at a pH between 4 and 10 and at a temperature between 40° C. and 70° C. degrees. Generally the enzymatic hydrolysis will be performed for a time comprised between 1 and 24 hours.

Optionally the native yeast enzymes are inactivated prior to the addition of any exogenous enzymes. Those skilled in the art know how to inactivate native yeast enzymes. Inactivation may for example be affected by a pH treatment or a heat shock, the latter method being preferred. A heat shock can be suitably performed by treating the yeast cell suspension at a temperature of 80-97° C. for 5 to 10 minutes. Once the native yeast enzymes have been inactivated, exogenous enzymes can be added to the suspension of yeast cells to perform the enzymatic hydrolysis. Preferably a protease, more preferably an endoprotease, is used for this purpose. Optionally an enzyme is used to transform RNA into 5′-ribonucleotides, like 5′-Fdase and optionally a deaminase, eg. adenylic deaminase, can also be used together with, or subsequently to, the treatment with the above-mentioned enzymes.

In a preferred embodiment the enzymatic hydrolysis is performed by subjecting the suspension of yeast cells to autolysis. Autolysis is a process wherein degradation of yeast cells and of polymeric yeast material is at least partially effected by active native yeast enzymes released in the medium after opening up of the yeast cells by (partially) damaging and/or disrupting the yeast cell wall.

Autolysis can be performed according to methods known in the art (for example, Conway J. et al, Can. J. Microbiol. (2001)47: 18-24).

Typically, autolysis of yeast cells is initiated by opening up of the yeast cells by (partially) damaging and/or disrupting yeast cell walls by mechanical, chemical or enzymatic treatments. Preferably, opening up of the yeast cells by (partially) damaging and/or disrupting the microbial cell wall is effected enzymatically. Several enzymes can be used, however a protease is preferably used, more preferably an endoprotease. Generally, the conditions used to open up the yeast cells and enzymatically damage and/or disrupt the microbial cell wall will correspond to those applied during the autolysis of the microorganism. When an enzyme is used to open up the yeast cells by (partially) damaging and/or disrupting the yeast cell wall, the enzyme may also contribute to the degradation of the yeast cells and of the polymeric yeast material.

Prior to step b), the enzyme(s) used in step a) may be generally inactivated e.g. using methods as mentioned above.

In step b) of the process to produce the mannoprotein used in the process of the invention, the mannoprotein solubilised and released from the degraded yeast cells is recovered. Preferably insoluble material, e.g. derived from yeast cell walls, is removed prior to recovery of the mannoprotein in step b), generally by a solid-liquid separation method, preferably by centrifugation or filtration. The mannoprotein may be recovered by any method suitable thereto. Preferably, the mannoprotein is recovered by ultrafiltration (UF). In cases where UF is used to recover mannoprotein, filters with a molecular weight cut-off of 100 kD or lower or preferably from 3 to 50 kD, more preferably from 3 to 10 kD, can be used. The mannoprotein fraction remains in the retentate resulting from the ultrafiltration step. When the insoluble material is not removed prior to ultrafiltration, the retentate comprising mannoprotein and the insoluble material can be resuspended (in solution) and the insoluble material is preferably removed.

Optionally, after step b) but prior to its use in the process to improve the activity as wine stabiliser, the recovered mannoprotein can be treated with Fdase to remove some RNA residues.

In the process according to the invention to improve the activity of a mannoprotein as wine stabiliser, a mannoprotein is treated with a basic solution at a pH of at least 9.

Preferably the treatment is performed at a pH of at least 10, preferably from 10 to 13, more preferably from 11 to 13. Generally the treatment with the basic solution is performed at a temperature between room temperature (e.g. 20° C.) and 120° C., more preferably between room temperature (e.g. 20° C.) and 100° C. Generally the treatment is performed for a period from 1 hour to 1 week, depending on the temperature. Generally, a higher pH will require a lower reaction temperature, while a higher reaction temperature will require a shorter reaction time. Therefore a treatment at e.g. pH 12 performed at room temperature for one week falls under the scope of the invention as well as a treatment at pH 12 and 70° C. for 2 hours or at pH 10 and 70° C. for 24 hours. Any suitable food-grade base can be used to perform the pH treatment. Examples of suitable bases are sodium or potassium hydroxide, sodium or potassium carbonate, sodium or potassium phosphate, or ammonium hydroxide. Sodium hydroxide or potassium hydroxide are preferred.

Preferably the treatment with the basic solution is performed under such conditions of temperature, duration and pH that the ³¹P-NMR of the product obtained after said treatment, measured in D₂O at a pH of 8, at 27° C. and using glycerophosphorylcholine (GPC) as an internal standard (the chemical shift value of GPC is taken as 0.43), shows the appearance or increase in intensity of one or more peaks between 4.5 and 5.5 ppm due to phosphomannan monoesters and the decrease in intensity or disappearance of one or more peaks between −1 and −2 ppm due to phosphomannan diesters when compared with the ³¹P-NMR spectrum, measured under the same conditions, of the mannoprotein before the treatment. Preferably the treatment with the basic solution is performed under conditions at which the ratio between the area of the one or more peaks between −1 and −2 ppm due to phosphomannan diesters and the area of the one or more peaks between 4.5 and 5.5 ppm due to phosphomannan monoesters in said ³¹P-NMR spectrum becomes at least 90:10, preferably at least 75:25, more preferably at least 50:50, even more preferably at least 25:75, even more preferably at least 10:90, most preferably approximately 0:100. Therefore most preferably the reaction is performed under conditions at which the one or more peaks between −1 and −2 ppm due to phosphomannan diesters (almost) completely disappear and are replaced by one or more peaks between 4.5 and 5.5 ppm due to phosphomannan monoesters. The man skilled in the art can e.g. distinguish peaks due to phosphomonoesters and phosphodiesters of phosphomannan from peaks due to other phosphomonoesters and phosphodiesters belonging to other compounds like e.g. RNA, mono-, oligo- and polyribonucleotides by two-dimensional NMR (e.g. by ³¹P-¹H correlation spectroscopy, see e.g. Chary K. V. R. et al J. Magn. Reson. Series B (1993) 102: 81-83).

In a preferred embodiment the treatment with the basic solution is performed under such conditions of temperature, duration and pH that also impurities due to RNA, oligo- and polyribonucleotides are at least in part, preferably at least for 50%, even more preferably completely degraded to monoribonucleotides. This degradation can be verified by ³¹P-NMR. The ³¹P-NMR of phoshodiesters due to RNA, oligo- and polyribonucleotides, measured under the same condition as above, comprises one or more peaks at ˜0 ppm while the ³¹P-NMR of phosphomonoesters due to monoribonucleotides comprises one or more peaks at ˜5 ppm, generally at about 4-5 ppm, at slightly higher fields than phosphomannan monoesters.

Optionally, once the treatment with the basic solution has been completed, the reaction mixture may be neutralised using food-grade acid known to those skilled in the art.

Preferably, the process of the invention further comprises the step of purifying the treated mannoprotein by ultrafiltration.

Typically this step is performed by subjecting the treated mannoprotein to one or more ultrafiltration steps. Ultrafiltration membranes with a molecular weight cut-off as indicated above can be used.

During the treatment with the basic solution some insolubles may be formed. In this case such insolubles can be eliminated by a common solid-liquid separation method such as filtration or centrifugation performed after the treatment with the basic solution but before the purification of the treated mannoprotein and/or performed after the purification of the treated mannoprotein.

The mannoprotein obtained with the process of the invention is generally obtained as a solution which can be further concentrated and/or dried by methods known in the art, e.g. by concentrating a mannoprotein solution under vacuum and by spray-drying or lyophilising the concentrated solution.

In a second aspect, the present invention provides a mannoprotein with improved activity as wine stabiliser obtainable by the process of the first aspect.

The mannoprotein according to the invention has preferably a molecular weight of at most 100 kDa, more preferably a molecular weight between 1-50 kDa, even more preferably between 3-30 kDa. The mannoprotein of the invention is preferably characterised by a carbohydrate content of at least 50% w/w, based on the mannoprotein dry matter, of which at least 70% w/w, based on the total carbohydrate content, consists of mannose residues in the form of mannose oligomers or polymers.

The ³¹P-NMR spectrum of the mannoprotein according to the invention, measured as indicated above, preferably comprises one or more peaks between −1 and −2 ppm due to phosphomannan diesters and/or one or more peaks between 4.5 and 5.5 ppm due to phospshomannan monoesters. More preferably the ratio between the area of the one or more peaks between −1 and −2 ppm due to phosphomannan diesters and the area of the one or more peaks between 4.5 and 5.5 ppm due to phospshomannan monoesters in said ³¹P-NMR spectrum is at least 90:10, preferably at least 75:25, more preferably at least 50:50, even more preferably at least 25:75, even more preferably at least 10:90, most preferably approximately 0:100.

Very surprisingly, the mannoprotein obtainable by the process of the invention has a higher activity as wine stabiliser if compared with the mannoprotein prior to the treatment with basic solution, i.e. when said mannoprotein is added in a sufficient amount to increase the T_{crys} of the treated wine or to decrease the turbidity of treated wine when compared with the untreated wine, said T_{crys} of the treated wine is longer or the turbidity of the treated wine is lower than the T_{crys} or the turbidity of the wine treated with the same amount of mannoprotein prior to the basic treatment.

It has been observed that an increased activity of the mannoprotein as wine stabiliser is correlated with an increased area of the peak(s) at 4.5-5.5 ppm due to phosphomannan monoesters in the ³¹P-NMR spectrum of the mannoprotein according to the invention and measured as indicated above.

The improved activity in wine of the mannoprotein according to the invention makes this mannoprotein especially suitable to be used as an additive in the stabilisation of wine.

The mannoprotein according to the invention can be used as sole additive to wine or in the form of a composition. Therefore in a third aspect the present invention provides a composition comprising the mannoprotein of the second aspect and one or more wine additives. Examples of wine additives are meta-tartrate or arabic gum. Preferred compositions comprise mannoprotein according to the invention and arabic gum.

In a fourth aspect the present invention provides the use of the mannoprotein of the second aspect or the composition of the third aspect in the stabilisation of wine.

In particular, the invention provides a process to stabilise wine by preventing and/or retarding the crystallisation of salts of tartaric acid wherein a mannoprotein according to the invention or a composition according to the invention is added to wine or to grape must to be used in the production of wine. The mannoprotein or compositions thereof is preferably added to the wine during ageing, i.e. after fermentation but before bottling. The invention is extremely suitable for white wines and rosé wines, but also for red wines.

The mannoprotein of the invention is added in a sufficient amount to achieve a stabilizing effect. Generally, the mannoprotein of the invention can be added to wine in a concentration between 10-1000 mg per liter of wine. Good results are already obtained by adding mannoprotein up to a final concentration in the wine of 10 to 400 mg per liter of wine. The skilled person will understand that the amount added will also depend on the addition or presence of e.g. other wine stabilisers and on the degree of supersaturation of the KHT in the wine prior to addition.

The nucleation and crystal growth of KHT in wine can be measured and quantified by the following methods (Moutounet et al. In: Actualités [symbol] nologiques 1999 Vieme Symposium International d'Oenologie de Bordeaux (Lonvaud-Funel ed.)).

The first method, indicative of crystal nucleation, measures the time of appearance of crystals in the wine when stored at −4° C. A visual inspection is performed daily and the time necessary to detect the appearance of crystals (T_{crys}) is expressed in number of days.

The second method, indicative of crystal growth, measures the Degree of Tartaric Instability (DTI) of the wine. Hereto, wines are stirred at −4° C. and the initial conductivity is measured. Subsequently, calibrated crystals of KHT are added and the conductivity is then measured after a stable value has been reached. The DTI is defined as the percentage decrease of the initial conductivity.

The third method measures the true, dissolved tartaric acid concentration. An accurate volume of the wine is transferred into a glass vial, and mixed with the same accurate volume of D₂O containing a precisely known concentration of maleic acid. The ¹H NMR spectrum is run with conditions of full relaxation, and the integral of the internal standard (maleic acid) is compared with the integral of tartaric acid. In this way the dissolved tartaric acid concentration can be determined with very high precision and accuracy.

The invention further provides a process to stabilise wine by preventing and/or reducing formation of protein haze wherein a mannoprotein according to the invention or a composition according to the invention is added to wine or to grape must to be used in the production of wine. Also in this case the mannoprotein of the invention is added in a sufficient amount to achieve a stabilizing effect. The stability of the wine in respect of protein haze formation after addition of the mannoprotein of the invention to the wine can be measured according to the following method. Wine samples are heated for 6 hours at 80° C. and then cooled down to 4° C. The induced haze due to unstable proteins is followed by turbidimetry (at 860 nm) or absorbance (at 540 nm) measurements.

Wine unstable in respect of crystallisation of tartaric acid salts has a T_{crys} that can vary between 0.5 and 15 days. Stabilized wine according to a further aspect of the invention is obtainable by adding to wine or to grape must to be used in fermentation for the production of wine the mannoprotein of the second aspect in a concentration suitable to prevent and/or retard the crystallization of salts of tartaric acid. Said stabilized wine is characterized by a T_{crys}^{stabilized wine}/T_{crys}^{unstable wine} of at least 2, preferably at least 5, more preferably at least 10, even more preferably between 20 and 40 as measured according to method 1.

Preferred features of one aspect of the invention are equally applicable, where appropriate, to another aspect.

The invention will now be illustrated by the following examples which do not intend however to be limiting.

### EXAMPLES

The amount of proteins in the mannoprotein of the invention can be determined by measuring the total nitrogen with the Kjeldahal method and by multiplying this value with the factor 6.25.

The amount of carbohydrates (based on mannoprotein dry matter) in the mannoprotein of the invention can be determined according to the well-known anthrone colorimetric method.

The amount of mannose in the mannoprotein of the invention can be measured using ion exchange chromatography. After hydrolysis of the mannoprotein with 4N TFA for 4 hours at 100° C., the hydrolysate is analysed, against pure mannose as a standard, using a CarboPac™ PA10 anion-exchange column (Dionex-USA) provided with an in-line pre-treatment AminoTrap™ column (Dionex-USA), a BorateTrap™ column (Dionex-USA) before the injection valve, and using an increasing gradient of NaOH. Detection of mannose is performed by using pulsed amperometric detection.

The amount of phosphorous in the mannoprotein can be measured according to a well-known AES-ICP method (Atomic Emission Spectroscopy with the aid of Inductive Coupled Plasma).

### Example 1

### Production of Mannoprotein from Yeast through an Autolytic Yeast Extraction Process

2 l of cream yeast from Saccharomyces cerevisiae was warmed up to 51° C. Subsequently 3.0 ml Pescalase® (commercially available serine protease from DSM Food Specialties, The Netherlands) was added and the mixture was incubated for 24 hours at pH 5.1, at 51.5° C. Next, the autolysate was heated for 1 hour at 65° C. to inactivate all enzyme activity. The extract (soluble fraction) was separated from the insoluble cell walls by means of centrifugation.

The high molecular weight mannoprotein, present in the soluble fraction were isolated from the other solubles by ultrafiltration over a filter with a cut-off of 10 kDa. The mannoprotein was recovered in the UF retentate fraction.

Data on the recovery of mannoprotein (MP-0) is presented in Table 1.

TABLE 1

| | | Dry | Dry |
|---|---|---|---|
| | Amount | matter | matter |
| Fraction | (g) | (%) | (g) |
| Cream yeast | 2000 | 18.0 | 360 |
| Autolysate | 2648 | 9.1 | 241 |
| UF retentate | 150 | 4.8 | 7.2 |
| (mannoprotein) | | | |

The ³¹ P-NMR of MP-0, measured under the conditions mentioned above, comprises a broad signal from δ +0.14 to δ −1.14 (polynucleotides), and two sharp signals at δ −1.33 and δ −1.40 (phosphodiesters of mannan).

### Example 2

### Basic Treatment of Mannoprotein Obtained in Example 1

A solution of crude mannoprotein, obtained by the process of Example 1, was prepared in a concentration of 20 g/l. The pH of the solution was adjusted to 12.0 with a 4M sodium hydroxyde solution, and the solution was stored at room temperature for 1 week. The pH was adjusted to 12.0 twice in the course of this period. After 1 week the solution was neutralized with a 4M hydrochloric acid solution. Finally, the salts and degradation product were removed by means of ultrafiltration using a membrane with a cut-off of 10 kD. The retentate (MP-1) was freeze-dried.

The ³¹P-NMR of MP-1 comprises two sharp signals at δ +5.13 and δ +5.01 (phosphomonoesters of mannan).

### Example 3

### Effect of Mannoprotein Obtained in Example 1 and 2 on the Crystallisation of KHT in Unstable Wine

The performance of MP-0 and MP-1 was compared.

MP-0 and MP-1 were dissolved in water in a concentration of 20 g/l. Small volumes were added to unstable white wine, to achieve final concentrations of 100, 150, 200, 300, 400 and 600 mg/l. Solutions were stored at −4° C.

Since MP-0 gave rise to unwanted haze, turbidity and precipitates when added to wine, after addition of the MP-0 mannoprotein solution to wine in the desired concentration the sample was stored for 2 hours at +4° C. During this period a significant precipitate developed which was removed by centrifugation. The clear supernatants of MP-0 was again stored at −4° C. All solutions were monitored on a daily basis for the appearance of crystals of KHT.

Table 2 summarizes the results presented as T_{crys} measured according to method 1 as described above.

Table 2 clearly shows that MP-1 is very effective as wine stabiliser when compared to MP-0. While wine treated with MP-0 shows formation of crystals of KHT only after a few days, the wine treated with MP-1 remains stable for more than 10 days even at low mannoprotein concentration. This experiment clearly demonstrates the efficiency of the process according to the invention in improving the activity of mannoprotein in preventing and/or retarding the crystallisation of salts of tartaric acid when the mannoprotein is added to wine or grape must to be used in the production of wine.

TABLE 2

| | |
|---|---|
| Concentration of | |
| mannoprotein | T_{crys} (days) |

| (mg/l) | MP-0 | | MP-1 | |
|---|---|---|---|---|
| 600 | 4 | | >25 | |
| 400 | 3 | | >25 | |
| 300 | 2 | | >25 | |
| 200 | 2 | | 17 | |
| 150 | 2 | | 11 | |
| 100 | 2 | | 10 | |
| 0 (blanc) | <16 | h | <16 | h |

### Example 4

### Characterisation of a Mannoprotein Obtained with the Method Described in Example 2

A mannoprotein obtained first as crude mannoprotein with the same method as described in example 1 and subsequently treated with the same method as described in example 2 was characterised for its content in carbohydrates, proteins and phosphorous. The results are reported in Table 3.

TABLE 3

| | % |
|---|---|
| Carbohydrates (Anthrone method) | 82.5 |
| Proteins (Nkj * 6.25) | 10.6 |
| Ashes | 3.9 |
| P | 0.34 |
| Water | 3.1 |
| Sum | 100.4 |
| Ratio mannose/other monosaccharides | 94 |

The amount of phosphorous, expressed as P₂O₅, corresponds to 0.77% w/w based on dry matter.

1. Process to improve the activity of a mannoprotein as wine stabiliser, the process comprising treating a mannoprotein with a basic solution at a pH of at least 9.

2. Process according to claim 1, which further comprises purifying the treated mannoprotein by ultrafiltration.

3. Process according to claim 1, wherein the treatment with a basic solution is performed at a pH of at least 10, preferably from 10 to 13, more preferably from 11 to 13.

4. Process according to claim 1, wherein the treatment with a basic solution is performed at a temperature from room temperature to 120° C., preferably from room temperature to 100° C.

5. Process according to claim 1, wherein the treatment with a basic solution is performed for a time from 1 hour to one week.

6. Process according to claim 1, wherein the treatment with the basic solution is performed under conditions of temperature, duration and pH at which the ³¹P-NMR of the product obtained after said treatment, measured in D₂O at a pH of 8, at 27° C., using glycerophosphorylcholine (GPC) as an internal standard and wherein the chemical shift value of GPC is taken as 0.43, shows the appearance or increase in intensity of one or more peaks between 4.5 and 5.5 ppm due to phosphomannan monoesters and the decrease in intensity or disappearance of one or more peaks between −1 and −2 ppm due to phosphomannan diesters when compared with the ³¹P-NMR spectrum, measured under the same conditions, of the mannoprotein before the treatment.

7. Process according to claim 6, wherein the treatment with the basic solution is performed under conditions at which the ratio between the area of the one or more peaks between −1 and −2 ppm due to phosphomannan diesters and the area of the one or more peaks between 4.5 and 5.5 ppm due to phosphomannan monoesters in said ³¹P-NMR spectrum becomes at least 90:10, preferably at least 75:25, more preferably 50:50, even more preferably at least 25:75, even more preferably at least 10:90, most preferably approximately 0:100.

8. Process according to claim 1 wherein the treatment with the basic solution is performed under such conditions of temperature, duration and pH that also impurities due to RNA, oligo- and polyribonucleotides are at least in part, preferably at least for 50%, even more preferably completely degraded to monoribonucleotides.

9. Mannoprotein with improved activity as wine stabiliser obtainable by a process according to claim 1.

10. Mannoprotein according to claim 9 wherein the 31P-NMR spectrum of the mannoprotein, measured in D₂O at a pH of 8, at 27° C., using glycerophosphorylcholine (GPC) as an internal standard wherein the chemical shift value of GPC is taken as 0.43, comprises one or more peaks between −1 and −2 ppm due to phosphomannan diesters and/or one or more peaks between 4.5 and 5.5 ppm due to phospshomannan monoesters, more preferably the ratio between the area of the one or more peaks between −1 and −2 ppm due to phosphomannan diesters and the area of the one or more peaks between 4.5 and 5.5 ppm due to phospshomannan monoesters in said ³¹P-NMR spectrum is at least 90:10, preferably at least 75:25, more preferably at least 50:50, even more preferably at least 25:75, even more preferably at least 10:90, most preferably approximately 0:100.

11. Composition comprising a mannoprotein according to claim 9 and one or more wine additives.

12. Process to stabilise wine comprising adding to wine a mannoprotein according to claim 9.

13. Process to stabilise wine by preventing or retarding the crystallisation of salts of tartaric acid comprising adding a stabilising effective amount of a mannoprotein according to claim 9 to wine or to grape used in the production of wine.

14. Process to stabilise wine by preventing and/or reducing formation of protein haze comprising adding a stabilising effective amount of a mannoprotein according to claim 9 to wine or to grape used in the production of wine.

15. Wine comprising a mannoprotein according to claim 9.
